(19) United States
(12) Patent Application Publication
(10) Pub. No.: US 2023/0116897 A1
(43) Pub. Date: Apr. 13, 2023

(54) SYSTEMS, METHODS, AND APPARATUSES FOR IMPLEMENTING SYSTEMATIC BENCHMARKING ANALYSIS TO IMPROVE TRANSFER LEARNING FOR MEDICAL IMAGE ANALYSIS

(71) Applicant: Arizona Board of Regents on behalf of Arizona State University, Scottsdale, AZ (US)

(72) Inventors: Mohammad Reza Hosseinzadeh Taher, Tempe, AZ (US); Fatemeh Haghighi, Tempe, AZ (US); Ruibin Feng, Scottsdale, AZ (US); Jianming Liang, Scottsdale, AZ (US)

(21) Appl. No.: 17/961,896

(22) Filed: Oct. 7, 2022

Related U.S. Application Data
(60) Provisional application No. 63/253,965, filed on Oct. 8, 2021.

(51) Int. Cl. G16H 50/20; G06T 7/00; G06V 10/774; G06V 10/82

(52) U.S. Cl. CPC G16H 50/20; G06T 7/0012; G06V 10/774; G06V 10/82

(57) ABSTRACT

Described herein are means for implementing systematic benchmarking analysis to improve transfer learning for medical image analysis. An exemplary system is configured with specialized instructions to cause the system to perform operations including: receiving training data having a plurality medical images therein; iteratively transforming a medical image from the training data into a transformed image by executing instructions for resizing and cropping each respective medical image from the training data to form a plurality of transformed images; applying data augmentation operations to the transformed images; applying segmentation operations to the augmented images; pre-training an AI model on different input images which are not included in the training data by executing self-supervised learning for the AI model; fine-tuning the pre-trained AI model to generate a pre-trained diagnosis and detection AI model; applying the pre-trained diagnosis and detection AI model to a new medical image to render a prediction as to the presence or absence of a disease within the new medical image; and outputting the prediction as a predictive medical diagnosis for a medical patient.

### CLAIM OF PRIORITY

This non-provisional U.S. Utility Patent Application is related to, and claims priority to the U.S. Provisional Patent Application No. 63/253,965, entitled “SYSTEMS, METHODS, AND APPARATUSES FOR IMPLEMENTING SYSTEMATIC BENCHMARKING ANALYSIS OF TRANSFER LEARNING FOR MEDICAL IMAGE ANALYSIS,” filed Oct. 8, 2021, having Attorney Docket Number 37684.673P, the entire contents of which are incorporated herein by reference as though set forth in full.

### GOVERNMENT RIGHTS AND GOVERNMENT AGENCY SUPPORT NOTICE

This invention was made with government support under R01 HL128785 awarded by the National Institutes of Health. The government has certain rights in the invention.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### TECHNICAL FIELD

Embodiments of the invention relate generally to the field of medical imaging and analysis using convolutional neural networks for the classification and annotation of medical images, and more particularly, to systems, methods, and apparatuses for implementing systematic benchmarking analysis to improve transfer learning for medical image analysis, in the context of processing of medical imaging.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to embodiments of the claimed inventions.

Machine learning models have various applications to automatically process inputs and produce outputs considering situational factors and learned information to improve output quality. One area where machine learning models, and neural networks in particular, provide high utility is in the field of processing medical images.

Within the context of machine learning and with regard to deep learning specifically, a Convolutional Neural Network (CNN, or ConvNet) is a class of deep neural networks, very often applied to analyzing visual imagery. Convolutional Neural Networks are regularized versions of multilayer perceptrons. Multilayer perceptrons are fully connected networks, such that each neuron in one layer is connected to all neurons in the next layer, a characteristic which often leads to a problem of overfitting of the data and the need for model regularization. Convolutional Neural Networks also seek to apply model regularization, but with a distinct approach. Specifically, CNNs take advantage of the hierarchical pattern in data and assemble more complex patterns using smaller and simpler patterns. Consequently, on the scale of connectedness and complexity, CNNs are on the lower extreme.

The present state of the art may therefore benefit from the systems, methods, and apparatuses for implementing systematic benchmarking analysis to improve transfer learning for medical image analysis, as is described herein.

### BRIEF DESCRIPTION OF THE DRAWINGS

Embodiments are illustrated by way of example, and not by way of limitation, and can be more fully understood with reference to the following detailed description when considered in connection with the figures in which:

FIGS. 1A, 1B, and 1C depict the effects of data granularity on transfer learning capability, in accordance with described embodiments;

FIG. 2 presents Table 1 showing how transfer learning was benchmarked for seven popular medical imaging tasks, spanning over different label structures (binary/multi-label classification and segmentation), modalities, organs, diseases, and data size, in accordance with described embodiments;

FIGS. 3A, 3B, 3C, 3D, 3E, 3F, and 3G show that for each target task, in terms of the mean performance, the supervised ImageNet model can be outperformed by at least three self-supervised ImageNet models, demonstrating the higher transferability of self-supervised representation learning, in accordance with described embodiments;

FIG. 4 presents Table 2 showing that domain-adapted pre-trained models outperform the corresponding ImageNet and in-domain models, in accordance with described embodiments;

FIG. 5 presents Table 3 showing the evaluation of iNat2021 mini dataset on segmentation medical tasks, according to described embodiments;

FIG. 6 presents Table 4 demonstrating benchmarking transfer learning from supervised iNat2021 and ImageNet models on seven medical tasks, according to described embodiments;

FIG. 7 presents Table 5 demonstrating benchmarking transfer learning from fourteen (14) self-supervised ImageNet pre-trained models on seven (7) medical tasks, according to described embodiments;

FIG. 8 presents Table 6 demonstrating that fine-tuning from the iNat2021 model provides higher performance in all segmentation tasks and considerably accelerates the training process in two out of three tasks in comparison to the ImageNet counterpart, according to described embodiments;

FIG. 9 presents Table 7 demonstrating that fine-tuning from the best self-supervised models provide significantly better or equivalent performance and accelerate the training process in comparison to the supervised counterpart, according to described embodiments;

FIG. 10 presents Table 8 demonstrating that fine-tuning from the domain-adapted pre-trained models provides higher performance in all tasks and speeds up the training process compared to the corresponding ImageNet models in most cases, in accordance with disclosed embodiments;

FIGS. 11A and 11B depict flow diagrams illustrating methods for implementing systematic benchmarking analysis to improve transfer learning for medical image analysis, in accordance with disclosed embodiments;

FIG. 12 shows a diagrammatic representation of a system within which embodiments may operate, be installed, integrated, or configured, in accordance with disclosed embodiments; and

FIG. 13 illustrates a diagrammatic representation of a machine in the exemplary form of a computer system, in accordance with one embodiment.

### DETAILED DESCRIPTION

Described herein are systems, methods, and apparatuses for implementing systematic benchmarking analysis to improve transfer learning for medical image analysis.

In the field of medical image analysis, transfer learning from supervised ImageNet models has been frequently used. And yet, no large-scale evaluation has been conducted to benchmark the efficacy of newly-developed pre-training techniques for medical image analysis, leaving several important questions unanswered. As the first step in this direction, a systematic study on the transferability of models pre-trained on iNat2021 was conducted, the most recent large-scale fine-grained dataset, and fourteen (14) top self-supervised ImageNet models on seven (7) diverse medical tasks in comparison with the supervised ImageNet model. Furthermore, devised and disclose herein is a practical approach to bridge the domain gap between natural and medical images by continually pre-training supervised ImageNet models on medical images. The disclosed comprehensive evaluation thus yields the following new insights: Firstly, pre-trained models on fine-grained data yield distinctive local representations that are more suitable for medical segmentation tasks. Secondly, self-supervised ImageNet models learn holistic features more effectively than supervised ImageNet models. And thirdly, continual pre-training has been demonstrated to bridge the domain gap between natural and medical images.

Through such innovations, it is further expected that large-scale open evaluation of transfer learning may additionally direct the future research of deep learning for medical imaging.

To circumvent the challenge of annotation dearth in medical imaging, fine-tuning supervised ImageNet models (e.g., models trained on ImageNet via supervised learning with the human labels) has become the standard practice. Nearly all top-performing models in a wide range of representative medical applications, including classifying the common thoracic diseases, detecting pulmonary embolism, identifying skin cancer, and detecting Alzheimer's disease, are fine-tuned from supervised ImageNet models. However, intuitively, achieving outstanding performance on medical image classification and segmentation would require fine-grained features. For instance, chest X-rays all look similar, therefore, distinguishing diseases and abnormal conditions may rely on some subtle image details.

Furthermore, delineating organs and isolating lesions in medical images would demand some fine-detailed features to determine the boundary pixels. In contrast to ImageNet, which was created for coarse-grained object classification, iNat2021, the most recent large-scale fine-grained dataset, has recently been created. It consists of 2.7M training images covering 10K species spanning the entire tree of life. As such, one may naturally ask the question: “What advantages can supervised iNat2021 models offer for medical imaging in comparison with supervised ImageNet models?”

In the meantime, numerous Self-Supervised Learning (SSL) methods have been developed. According to the various embodiments specific to the use of transfer learning, models are pre-trained in a supervised manner using expert-provided labels. By comparison, SSL pre-trained models use machine-generated labels. The recent advancement in Self-Supervised Learning has resulted in self-supervised pre-training techniques that surpass gold standard supervised ImageNet models in a number of computer vision tasks. Therefore, a second question that may be raised asks: “How generalizable are the self-supervised ImageNet models to medical imaging in comparison with supervised ImageNet models?”

More importantly, there are significant differences between natural and medical images. Medical images are typically monochromic and consistent in anatomical structures. Now, several moderately-sized datasets have been created in medical imaging, for instance, NIH ChestX-Ray14 which includes 112K images and CheXpert which consists of 224K images. Naturally, a third question is therefore: “Can these moderately-sized medical image datasets help bridge the domain gap between natural and medical images?”

To answer these questions, the first extensive benchmarking study was formulated and conducted to evaluate the efficacy of different pre-training techniques for diverse medical imaging tasks, covering various diseases (e.g., embolism, nodule, tuberculosis, etc), organs (e.g., lung and fundus), and modalities (e.g., CT, X-ray, and fundoscopy).

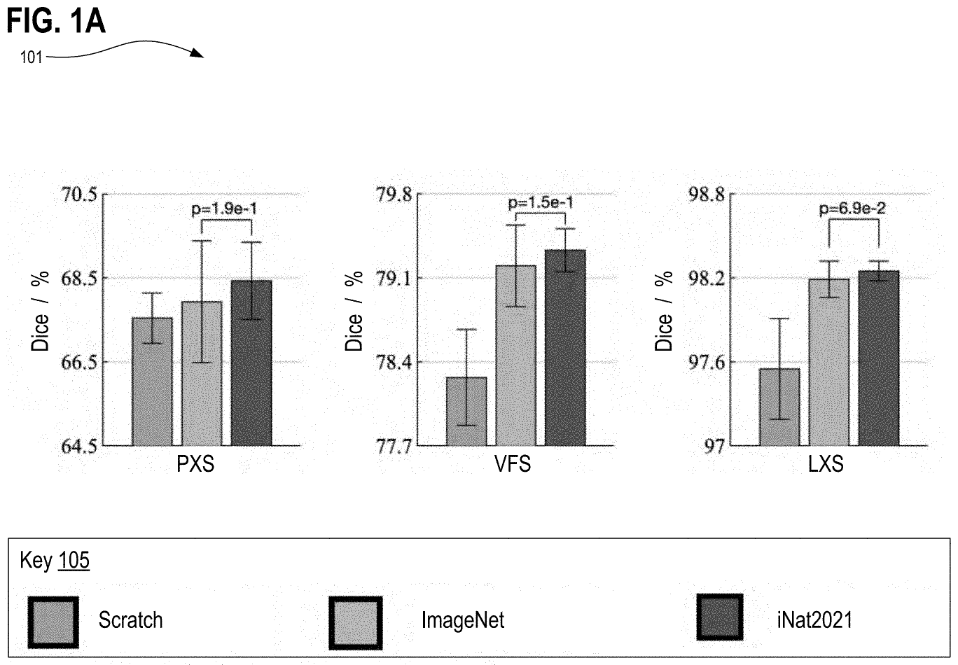

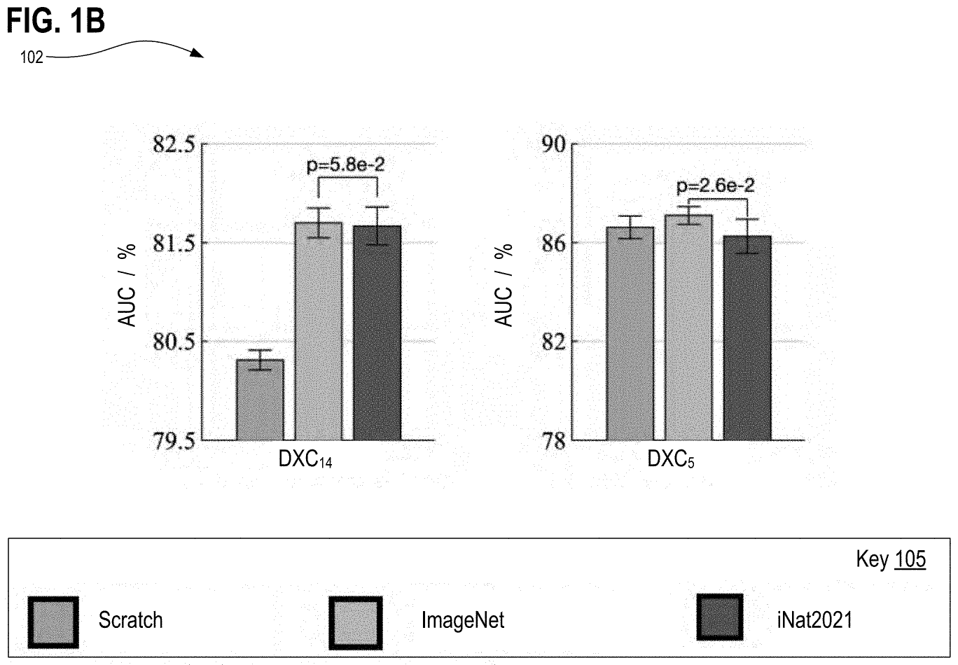

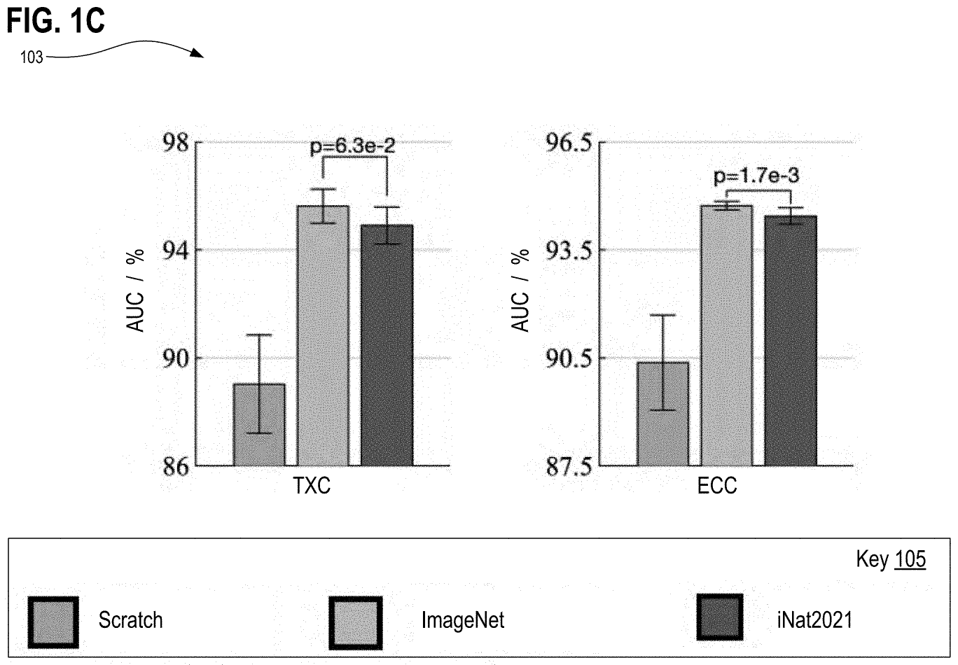

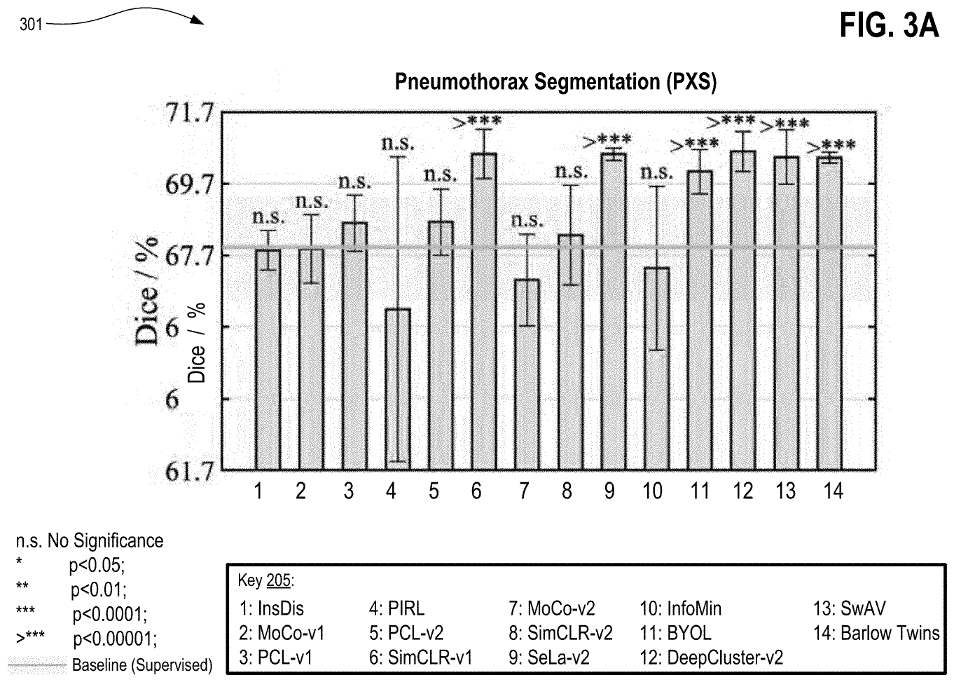

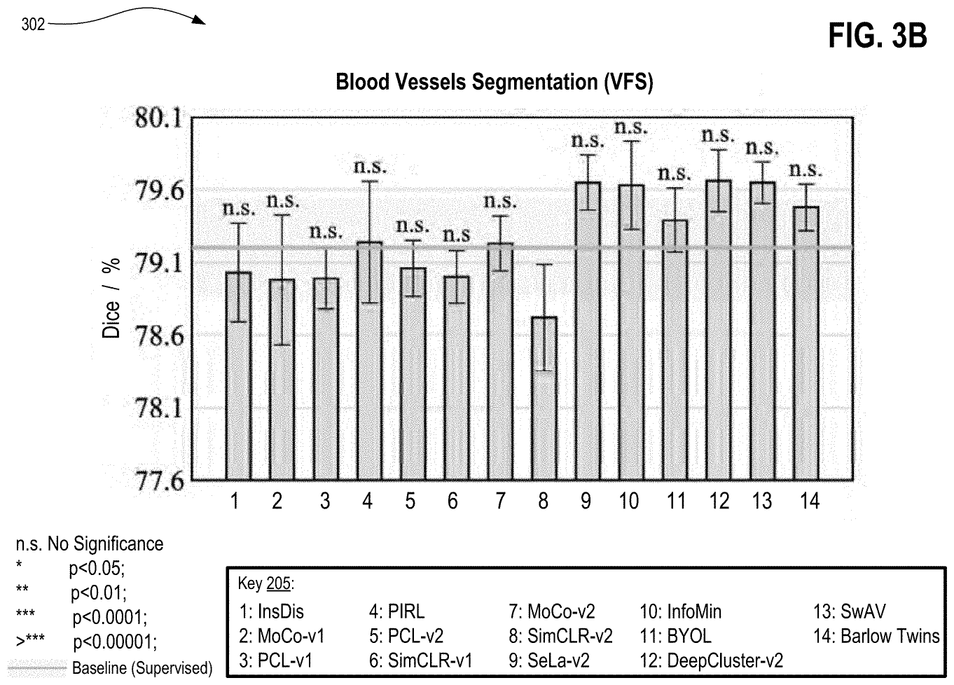

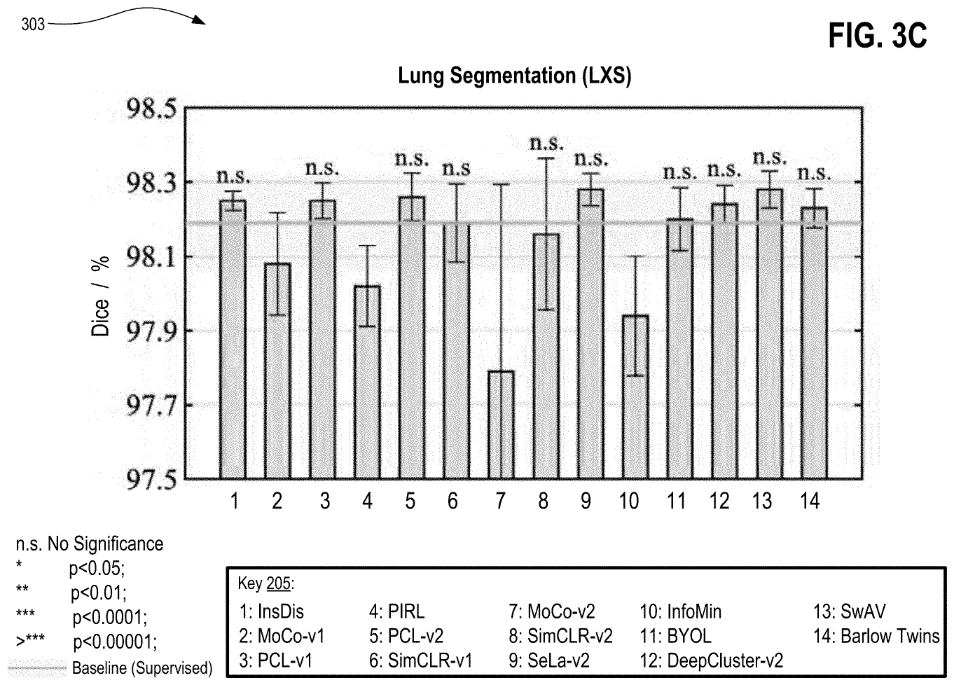

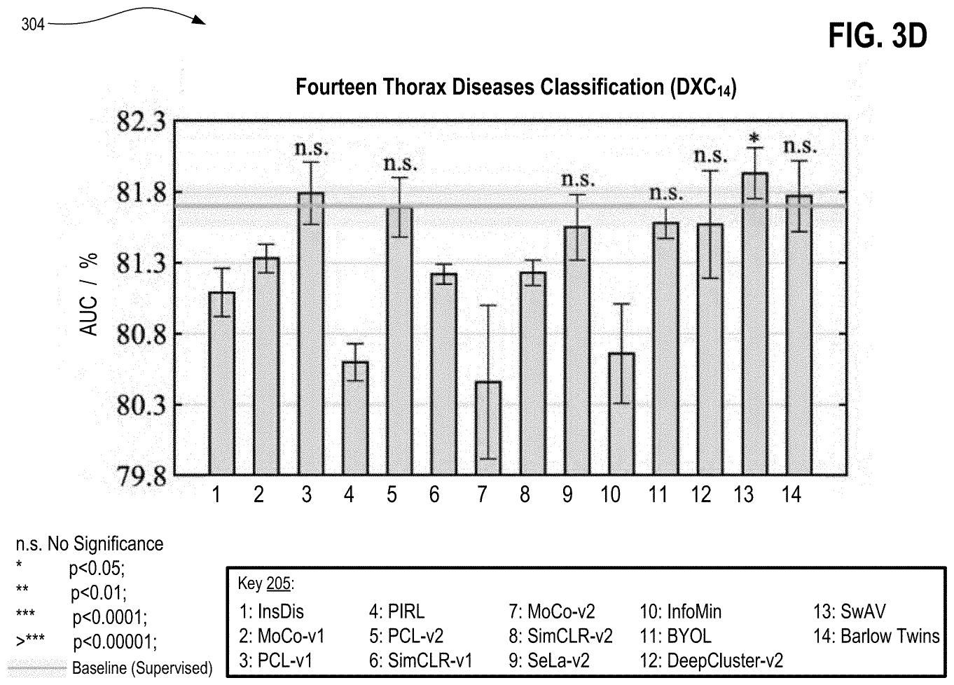

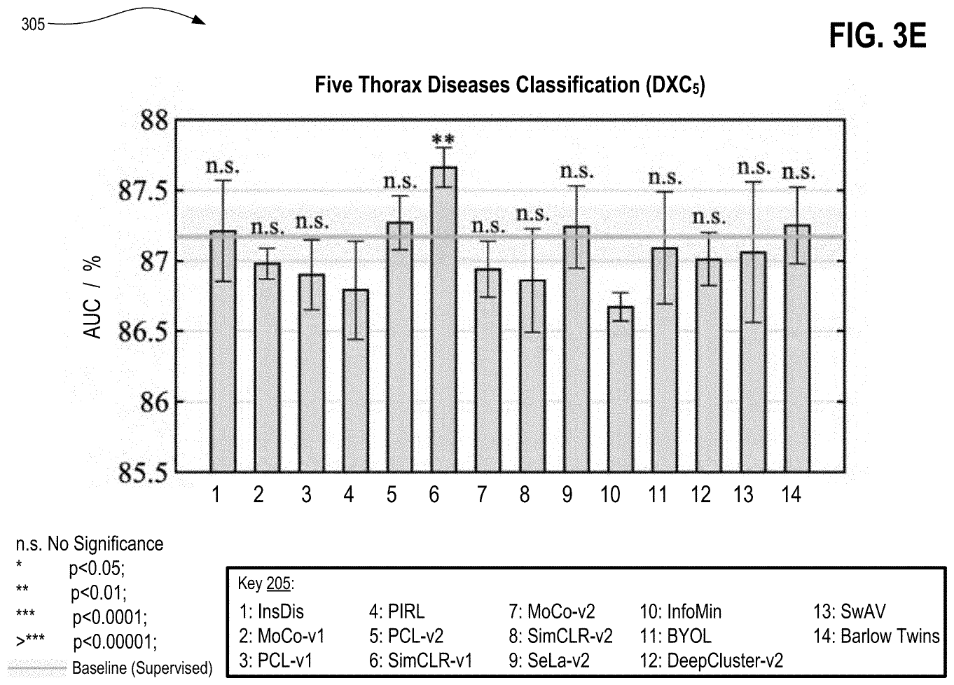

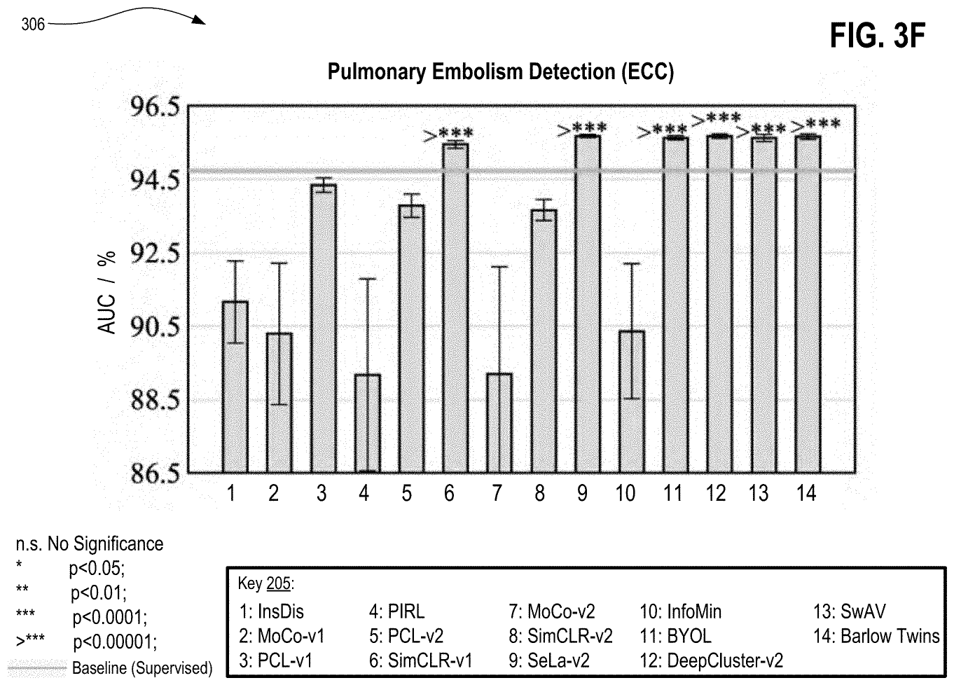

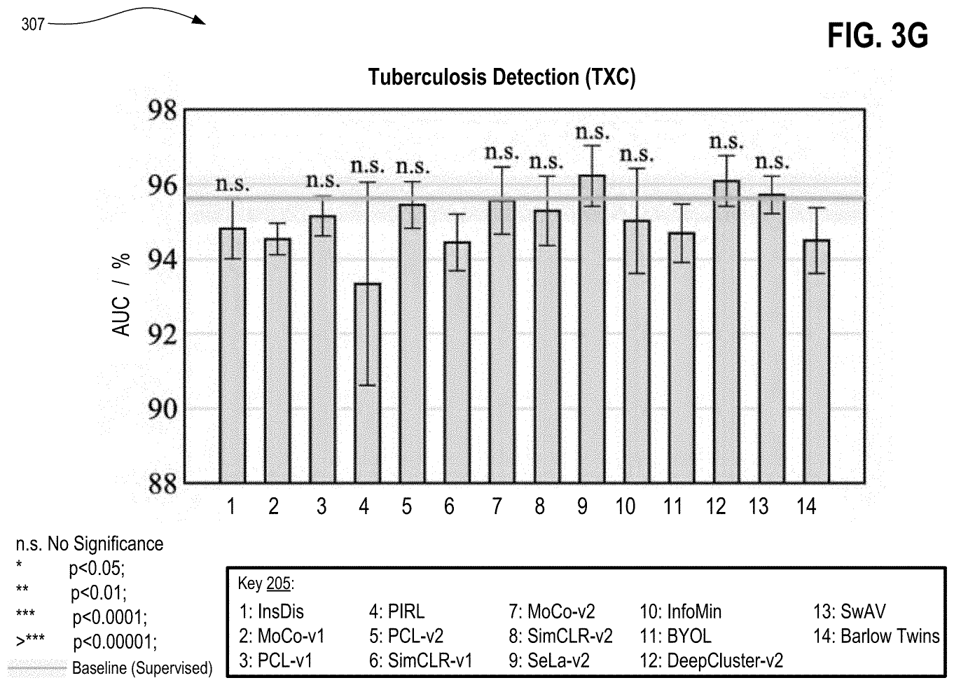

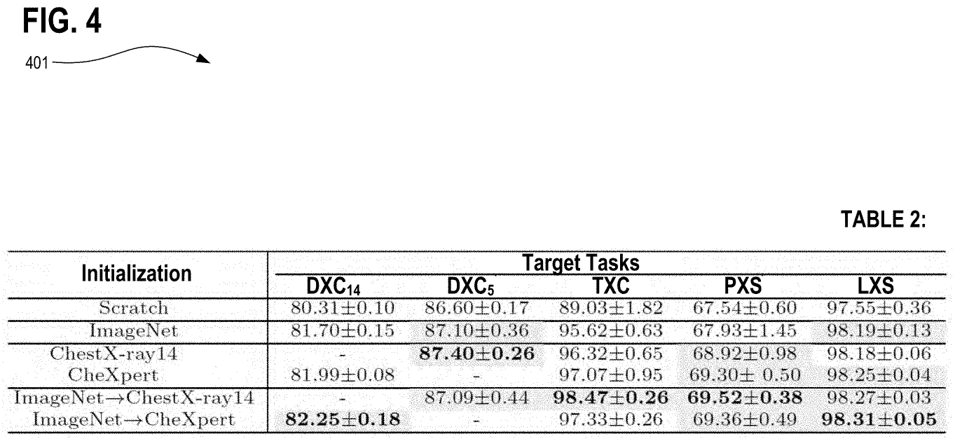

Specifically studied was the impact of pre-training data granularity on transfer learning performance by evaluating the fine-grained pre-trained models on iNat2021 for various medical tasks. Secondly, the transferability of fourteen (14) state-of-the-art self-supervised ImageNet models was evaluated against a diverse set of tasks in medical image classification and segmentation. Thirdly, domain-adaptive (continual) pre-training on natural and medical datasets was evaluated to tailor ImageNet models for target tasks on chest X-rays. The extensive empirical study revealed the following important insights: First, pre-trained models on fine-grained data yield distinctive local representations that are beneficial for medical segmentation tasks, while pre-trained models on coarser-grained data yield high-level features that prevail in classification target tasks (refer to FIGS. 1A, 1B, and 1C below). Second, for each target task, in terms of the mean performance, there exist at least three self-supervised ImageNet models that outperform the supervised ImageNet model, an observation that is very encouraging, as migrating from conventional supervised learning to self-supervised learning will dramatically reduce annotation efforts (refer to FIGS. 3A, 3B, 3C, 3D, 3E, 3F, and 3G below). Third, continual pre-training of supervised ImageNet models on medical images can bridge the gap between the natural and medical domains, providing more powerful pre-trained models for medical tasks (refer to Table 2 as set forth at FIG. 4 below).

FIGS. 1A, 1B, and 1C depict the effects of data granularity on transfer learning capability, in accordance with described embodiments.

More particularly, the results shown here demonstrate that for segmentation (target) tasks (e.g., PXS, VFS, and LXS as depicted at FIG. 1A, element 101), fine-tuning the model pre-trained on iNat2021 outperforms that on ImageNet, while the model pre-trained on ImageNet prevails on classification (target) tasks (e.g., DXC₁₄, and DXC₅ as depicted at FIG. 1B, element 102, and TXC, and ECC as depicted at FIG. 1C, element 103), demonstrating the effect of data granularity on transfer learning capability: pre-trained models on the fine-grained data capture subtle features that empowers segmentation target tasks, and pre-trained models on the coarse-grained data encode high-level features that facilitate classification target tasks.

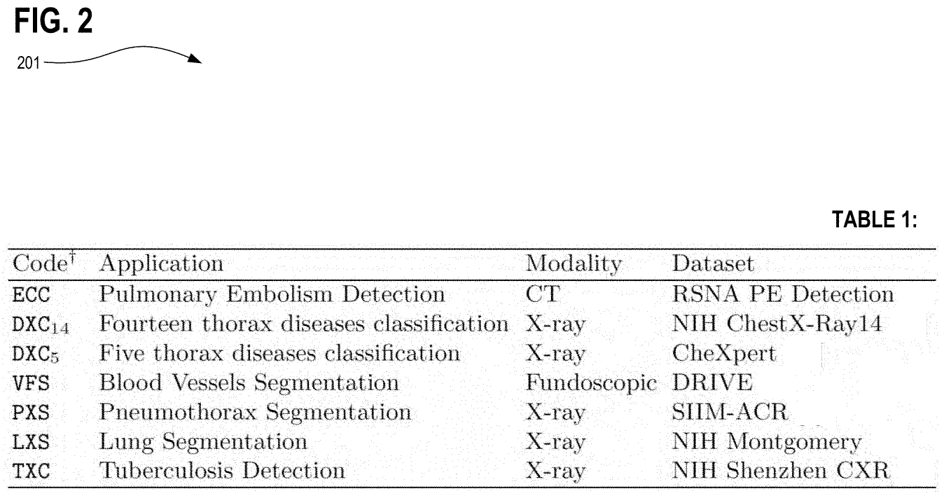

FIG. 2 presents Table 1 (element 201) showing how transfer learning was benchmarked for seven popular medical imaging tasks, spanning over different label structures (binary/multi-label classification and segmentation), modalities, organs, diseases, and data size, in accordance with described embodiments.

### Transfer Learning Setup

Tasks and datasets—Table 1 summarizes the tasks and datasets. Detailed results are provided below with reference to Tables 3, 4, 5, 6, 7, and 8 as set forth at FIGS. 4 through 10.

A diverse suite of seven (7) challenging and popular medical imaging tasks was considered, covering various diseases, organs, and modalities. These tasks span many common properties of medical imaging tasks, such as imbalanced classes, limited data, and small-scanning areas of pathologies of interest. The official data split of these datasets was utilized when available. Otherwise, the datasets were randomly divided into 80%/20% for training/testing, respectively.

Evaluations—Various models pre-trained with different methods and datasets were evaluated which enabled control over other influencing factors such as preprocessing, network architecture, and transfer hyperparameters. In all experiments: (1) for the classification target tasks, the standard ResNet-50 backbone followed by a task-specific classification head was used, (2) for the segmentation target tasks, a U-Net network with a ResNet-50 encoder was used, where the encoder is initialized with the pre-trained models, (3) all target model parameters are fine-tuned, (4) AUC (area under the ROC curve) and Dice coefficient were used for evaluating classification and segmentation target tasks, respectively, (5) mean and standard deviation of performance metrics over ten runs were reported, and (6) statistical analyses based on independent two-sample t-test were presented.

Detailed results are provided below with respect to Tables 3 through 8 as set forth at FIGS. 4 through 10.

Pre-trained models—For the sake of experiment, transfer learning was benchmarked from two large-scale natural datasets, ImageNet and iNat2021, and two in-domain medical datasets, CheXpert and ChestX-Ray14. Supervised in-domain models were pre-trained, which were either initialized randomly or fine-tuned from the ImageNet model. For all other supervised and self-supervised methods, existing official and ready-to-use pre-trained models were used, thus ensuring that their configurations were meticulously assembled to achieve the best results in target tasks.

Transfer Learning Benchmarking and Analysis—Pre-trained models on fine-grained data are better suited for segmentation tasks, while pre-trained models on coarse-grained data prevail on classification tasks. Medical imaging literature mostly has focused on the pre-training with coarse-grained natural image datasets, such as ImageNet. In contrast to previous works, experiments aimed to study the capability of pre-training with fine-grained datasets for transfer learning to medical tasks. In fine-grained datasets, visual differences between subordinate classes are often subtle and deeply embedded within local discriminative parts. Therefore, a model should capture visual details in the local regions for solving a fine-grained recognition task. It was hypothesized that a pre-trained model on a fine-grained dataset derives distinctive local representations that are useful for medical tasks which usually rely upon small, local variations in texture to detect/segment pathologies of interest. To put this hypothesis to the test, experiments empirically validated how well pre-trained models on large-scale fine-grained datasets can transfer to a range of target medical applications. This study represents the first effort to rigorously evaluate the impact of pre-training data granularity on transfer learning to medical imaging tasks.

Experimental setup—The applicability of iNat2021 was examined as a pre-training source for medical imaging tasks. The goal was to compare the generalization of the learned features from fine-grained pre-training on iNat2021 with the conventional pre-training on the ImageNet. Given this goal, the existing official and ready-to-use pre-trained models were utilized on these two datasets, and they were fine-tuned for seven (7) diverse target tasks, covering multi-label classification, binary classification, and pixel-wise segmentation (refer again to Table 1 at FIG. 2). To provide a comprehensive evaluation, results for training target models from scratch were additionally included.

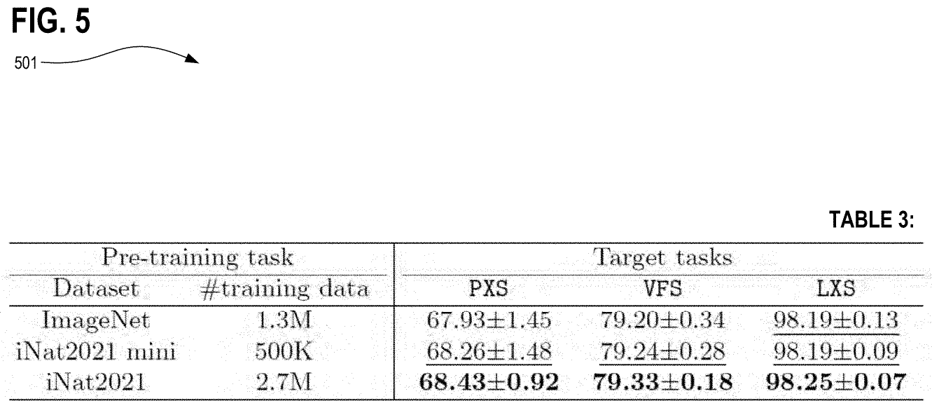

Observations and Analysis—As evidenced by FIG. 1A, fine-tuning from the iNat2021 pre-trained model outperforms the ImageNet counterpart in semantic segmentation tasks, e.g., PXS, VFS, and LXS (refer to FIG. 1A, element 101). This implies that, owing to the finer data granularity of iNat2021, the pre-trained model on this dataset yields a more fine-grained visual feature space, which captures essential pixel-level cues for medical segmentation tasks. This observation gives rise to a natural question of whether this improved performance can be attributed to the larger pre-training data of iNat2021 (2.7M images) compared to ImageNet (1.3M images). In answering this question, an ablation study was conducted on the iNat2021 mini dataset with 500K images to further investigate the impact of data granularity on the learned representations. Results demonstrate that even with fewer pre-training data, iNat2021 mini pre-trained models can outperform ImageNet counterparts in segmentation tasks (refer to the results in Table 3 as set forth at FIG. 5 below). The results thus demonstrate that recovering discriminative features from iNat2021 dataset should be attributed to fine-grained data rather than the larger training data size.

Despite the success of iNat2021 models in segmentation tasks, fine-tuning of ImageNet pre-trained features outperforms iNat2021 in classification tasks, namely DXC₁₄, DXC₅ (refer again to FIG. 1B), TXC, and ECC (refer again to FIG. 1C). Contrary to initial expectations, pre-training on a coarser granularity dataset, such as ImageNet, yields high-level semantic features that are more beneficial for classification tasks.

Generally speaking, fine-grained pre-trained models could be a viable alternative for transfer learning to fine-grained medical tasks, hoping practitioners will find this observation useful in migrating from standard ImageNet checkpoints to reap the benefits demonstrated here. Regardless of, or perhaps in addition to, other advancements, visually diverse datasets like ImageNet can continue to play a valuable role in building performant medical imaging models.

Self-supervised ImageNet models outperform supervised ImageNet models—A recent family of self-supervised ImageNet models has demonstrated superior transferability in an increasing number of computer vision tasks compared to supervised ImageNet models. Self-supervised models, in particular, capture task-agnostic features that can be easily adapted to different domains, while high-level features of supervised pre-trained models may be extraneous when the source and target data distributions are far apart. It is hypothesized that this phenomenon is more pronounced in the medical domain, where there is a remarkable domain shift when compared to ImageNet. To test this hypothesis, the effectiveness of a wide range of recent self-supervised methods were dissected, encompassing contrastive learning, clustering, and redundancy-reduction methods, on the broadest benchmark yet of various modalities spanning X-ray, CT, and fundus images. This work represents the first effort to rigorously benchmark SSL techniques to a broader range of medical imaging problems.

Experimental setup—Transferability of fourteen (14) popular SSL methods were evaluated with officially released models, which had been expertly optimized, including contrastive learning (CL) based on instance discrimination (e.g., InsDis, MoCo-v1, MoCo-v2, SimCLR-v1, SimCLR-v2, and BYOL), CL based on JigSaw shuffling (PIRL), clustering (DeepCluster-v2 and SeLa-v2), clustering bridging CL (PCL-v1, PCL-v2, and SwAV), mutual information reduction (InfoMin), and redundancy reduction (Barlow Twins), on seven (7) diverse medical tasks.

All methods were pre-trained on the ImageNet and use ResNet-50 architecture. Details of SSL methods can be found below (with reference to the section labeled “Self-supervised Learning Methods”). As the baseline, the standard supervised pre-trained model was considered on ImageNet with a ResNet-50 backbone.

FIGS. 3A, 3B, 3C, 3D, 3E, 3F, and 3G show that for each target task, in terms of the mean performance, the supervised ImageNet model can be outperformed by at least three self-supervised ImageNet models, demonstrating the higher transferability of self-supervised representation learning, in accordance with described embodiments.

Recent approaches, SwAV, Barlow Twins, SeLa-v2, and DeepCluster-v2, stand out as consistently outperforming the supervised ImageNet model in most target tasks. Statistical analysis was conducted between the supervised model and each self-supervised model in each target task, and show the results for the methods that significantly outperform the baseline or provide comparable performance. Methods are listed in numerical order from left to right.

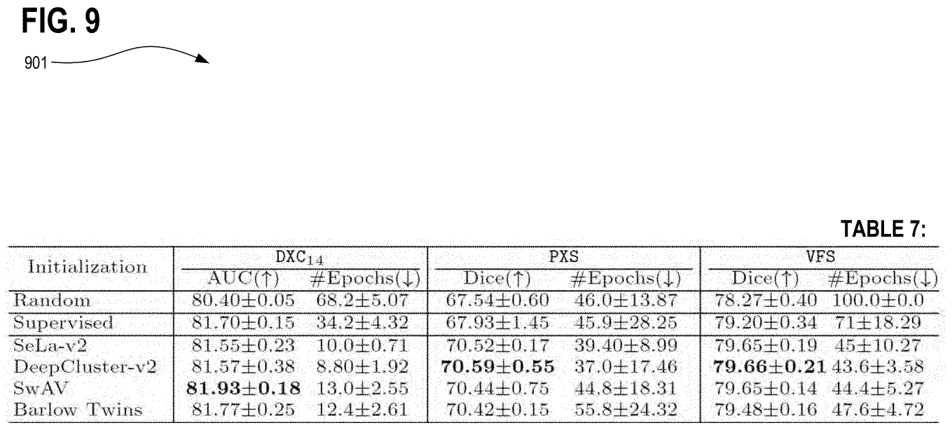

Observations and Analysis—According to FIGS. 3A, 3B, 3C, 3D, 3E, 3F, and 3G, for each target task, there are at least three self-supervised ImageNet models that outperform the supervised ImageNet model on average. Moreover, the top self-supervised ImageNet models remarkably accelerate the training process of target models in comparison with a supervised-learning counterpart (refer to Table 7 as set forth at FIG. 9 below). Intuitively, supervised pre-training labels encourage the model to retain more domain-specific high-level information, causing the learned representation to be biased toward the pre-training task/dataset's idiosyncrasies. Self-supervised learners, however, capture low/mid-level features that are not attuned to domain-relevant semantics, generalizing better to diverse sorts of target tasks with low-data regimes.

Comparing the classification (DXC₁₄, DXC₅, ECC, and TXC as set forth at FIG. 3D element 304, FIG. 3E element 305, FIG. 3F element 306, and FIG. 3G element 307, respectively) and segmentation tasks (PXS, VFS, and LXS as set forth at FIG. 3A element 301, FIG. 3B element 302, and FIG. 3C element 303, respectively), in the latter segmentation tasks, a larger number of SSL methods results in better transfer performance, while supervised pre-training falls short. This suggests that when there are larger domain shifts, self-supervised models can provide more precise localization than supervised models. This is because supervised pre-trained models primarily focus on the smaller discriminative regions of the images, whereas SSL methods attune to larger regions, which empowers them with deriving richer visual information from the entire image.

Thus, generally speaking, SSL can learn holistic features more effectively than supervised pre-training, resulting in higher transferability to a variety of medical tasks. Notably, no single SSL method dominates in all tasks, implying that universal pre-training remains a mystery. It is expected that the results of this benchmarking may resonate with recent studies in the natural image domain, thus leading to more effective transfer learning for medical image analysis.

Domain-adaptive pre-training bridges the gap between the natural and medical imaging domains—Pre-trained ImageNet models are the predominant standard for transfer learning as they are free, open-source models which can be used for a variety of tasks. Despite the prevailing use of ImageNet models, the remarkable covariate shift between natural and medical images restrain transfer learning. This constraint motivates us to present a practical approach that tailors ImageNet models to medical applications. Towards this end, experiments investigate domain-adaptive pre-training on natural and medical datasets to tune ImageNet models for medical tasks.

Experimental Setup—The domain-adaptive paradigm originated from natural language processing. This is a sequential pre-training approach in which a model is first pre-trained on a massive general dataset, such as ImageNet, and then pre-trained on domain-specific datasets, resulting in domain-adapted pre-trained models. For the first pre-training step, the supervised ImageNet model was used. For the second pre-training step, two new models were created each of which being initialized through the ImageNet model followed by supervised pre-training on CheXpert (ImageNet→CheXpert) and ChestX-ray14 (ImageNet→ChestX-ray14). The domain-adapted models were then compared with (1) the ImageNet model, and (2) two supervised pre-trained models on CheXpert and ChestX-ray14, which are randomly initialized. In contrast to previous methodologies which are limited to two classification tasks, domain-adapted models were evaluated on a broader range of five target tasks on chest X-ray scans. These tasks span classification and segmentation, ascertaining the generality of the disclosed findings.

FIG. 4 presents Table 2 (element 401) showing that domain-adapted pre-trained models outperform the corresponding ImageNet and in-domain models, in accordance with described embodiments.

For every target task, the independent two sample t-test was performed between the best (bolded) vs. others. Highlighted boxes in green indicate results which have no statistically significant difference at the p=0.05 level. When pre-training and target tasks are the same, transfer learning is not applicable, denoted by the dash (-) symbol. The footnotes compare the disclosed results with the state-of-the-art performance for each task.

Observations and Analysis—From the results set forth at FIG. 4, the following observations are drawn: First, both ChestX-ray14 and CheXpert models consistently outperform the ImageNet model in all cases. This observation implies that in-domain medical transfer learning, whenever possible, is preferred over ImageNet transfer learning. The conclusion here is opposite to prior methodologies and techniques, where in-domain pre-trained models outperform ImageNet models in controlled setups but lag far behind the real-world ImageNet models. Second, the overall trend showcases the advantage of domain-adaptive pre-training. Specifically, for DXC₁₄, fine-tuning the ImageNet→CheXpert model surpasses both ImageNet and CheXpert models. Furthermore, the dominance of domain-adapted models (ImageNet→CheXpert and ImageNet→ChestX-ray14) over ImageNet and corresponding in-domain models (CheXpert and ChestX-ray14) is conserved at LXS, TXC, and PXS. This suggests that domain-adapted models leverage the learning experience of the ImageNet model and further refine it with domain-relevant data, resulting in more pronounced representation. Third, in DXC₅, the domain-adapted performance decreases relative to corresponding ImageNet and in-domain models. This is most likely due to the lesser number of images in the in-domain pre-training dataset than the target dataset (75K vs. 200K), suggesting that in-domain pre-training data should be larger than the target data.

Thus, generally speaking, continual pre-training can bridge the domain gap between natural and medical images. Concretely, the readily conducted annotation efforts were leveraged to produce more performant medical imaging models and reduce future annotation burdens. It is expected that findings demonstrated here will posit new research directions for developing specialized pre-trained models in medical imaging.

Thus, described herein is the first fine-grained and up-to-date study on the transferability of various brand-new pre-training techniques for medical imaging tasks, answering central and timely questions on transfer learning in medical image analysis. The empirical evaluation suggests that: (1) what truly matters for the segmentation tasks is fine-grained representation rather than high-level semantic features, (2) top self-supervised ImageNet models outperform the supervised ImageNet model, offering a new transfer learning standard for medical imaging, and (3) ImageNet models can be strengthened with continual in-domain pre-training.

As described herein, transfer learning from the supervised ImageNet model as the baseline has been considered, upon which all evaluations are benchmarked. To compute p-values for statistical analysis, fourteen (14) self-supervised-learning, five (5) supervised, and two (2) domain-adaptive pre-trained models were run ten (10) times each on a set of seven (7) target tasks, thus leading to a large number of experiments (1,420 in total). Nevertheless, the self-supervised models were all pre-trained on ImageNet with ResNet50 as the backbone. While ImageNet is generally regarded as a strong source for pre-training, pre-training modern self-supervised models with iNat2021 and in-domain medical image data on various architectures may offer even deeper insights into transfer learning for medical imaging.

### Datasets

iNat2021: The iNaturalist2021 dataset (iNat2021) is a recent large-scale, fine-grained species dataset with 2.7M training images covering 10k species. This dataset facilitates fine-grained visual classification problems. Compared to the more widely used dataset, ImageNet, iNat2021 contains a greater number of these fine-grained images but a narrower range of visual diversity.

iNat2021 mini In addition to the full-sized dataset, a smaller version of iNat2021 was created, named iNat2021 mini, that contains 50 training images per species, sampled from the full train split. In total, iNat2021 mini includes 500K training images covering 10k species.

ChestX-ray14: This hospital-scale chest X-ray dataset contains 112K frontal-view X-ray images taken from a sample of 30K unique patients. ChestX-ray14 provides an official patient-wise split for training (86K images) and test sets (25K images). In this dataset, 51K images have at least one of the 14 thorax diseases. Experiments described herein utilized the official data split and report the mean AUC score over fourteen (14) diseases for the multi-label chest X-ray classification task.

CheXpert: This large-scale publicly available dataset contains 224K high-quality chest X-ray images taken from a sample of 65K patients. The training images were annotated by a labeler to automatically detect the presence of fourteen (14) thorax diseases in radiology reports, capturing uncertainties inherent in radiograph interpretation. The test set consists of 234 images from 200 patients. The test images were manually annotated by board-certified radiologists for 5 selected diseases, e.g., Cardiomegaly, Edema, Consolidation, Atelectasis, and Pleural Effusion. Experiments described herein utilized the official data split and report the mean AUC score over five (5) test diseases.

SIIM-ACR PS-2019: The Society for Imaging Informatics in Medicine (SIIM) and American College of Radiology provided the SIIM-ACR Pneumothorax Segmentation dataset, consisting of 10K chest X-ray images and the segmentation masks for Pneumothorax disease. The experiments described herein divided the dataset into training (80%) and testing (20%), and the segmentation performance was evaluated by using the Dice coefficient score.

RSNA PE Detection: This dataset is the largest publicly available annotated Pulmonary Embolism (PE) dataset, comprised of more than 7,000 CT scans with a varying number of images in each scan. Each image has been annotated for the presence or absence of the PE. Also, each scan has been labeled for additional nine patient-level labels. The experiments described herein randomly split the data at patient-level to training (6K) and testing (1K) sets, respectively. Correspondingly, there are 1.5M and 248K images in the training and testing sets, respectively. The AUC score is reported for the PE detection task.

NIH Shenzhen CXR: The dataset contains 662 frontal-view chest X-rays, of which 326 are normal cases and 336 are cases with manifestations of Tuberculosis (TB), including pediatric X-rays (AP). The experiments described herein randomly divide the dataset into a training set (80%) and a test set (20%). The AUC score is reported for the Tuberculosis detection task.

NIH Montgomery: The dataset contains 138 frontal-view chest X-rays from Montgomery County's Tuberculosis screening program, of which 80 are normal cases and 58 are cases with manifestations of TB. The segmentation masks for left and right lungs are provided. The experiments described herein randomly divided the dataset into a training set (80%) and a test set (20%) and report the mean Dice score for the lung segmentation task.

DRIVE: The dataset contains 40 retinal images, separated by its providers into a training set (20 images) and a test set (20 images). For all images, manual segmentation of the vasculature is provided. Experiments described herein use the official data split and report the mean Dice score for the segmentation of blood vessels.

### Implementation

Experiments evaluated popular publicly available representations that have been pre-trained with various methods and datasets across a variety of target tasks, thus permitting control over other influencing factors such as pre-processing, network architecture, and transfer hyperparameters. Experimental results were obtained by running each method ten times on all of the target tasks and reporting the average, standard deviation, and then further presenting statistical analysis based on an independent two-sample t-test.

Architecture—The network architecture was fixed in all experiments so as to understand the competitiveness of representations rather than benefits from varying specialized architecture. Therefore, all the pre-trained models leveraged the same ResNet-50 backbone. For transfer learning to the classification target tasks, the pre-trained ResNet-50 models were taken and a task-specific classification head was appended. For the segmentation target tasks, experiments utilized a U-Net network with a ResNet-50 encoder, where the encoder is initialized with the pre-trained models. Further evaluated was the transfer learning performance of all pre-trained models by fine-tuning all layers in the downstream networks.

Preprocessing and data augmentation—For target tasks on X-ray modality (DXC₁₄, DXC₅, TXC, LXS, and PXS), Fundoscopic modality (VFS), and CT modality (ECC), for the purposes of experimentation, the images were resized to 224×224, 512×512, and 576×576, respectively. For all classification target tasks, standard data augmentation techniques were applied, including random cropping, horizontal flipping, and rotating. For segmentation tasks on X-ray modality (LXS and PXS), RandomBrightnessContrast, RandomGamma, OpticalDistortion, elastic transformation, and grid distortion were employed. For segmentation task on fundoscopic modality (VFS), random rotation, Gaussian noise, color jittering, and horizontal, vertical and diagonal flips were utilized.

Training parameters—Since different datasets require different optimal settings, each target task was optimized with the best performing hyperparameters. In all experiments, an Adam optimizer was utilized with β¹=0.9, and β₂=0.999.

Further utilized were ReduceLROnPlateau and cosine learning rate decay schedulers for classification and segmentation tasks, respectively. If no improvement was seen in the validation set for a certain number of epochs, then the learning rate was reduced. Early-stop mechanisms were utilized using the 10% of the training data as the validation set to avoid over-fitting.

For X-ray classification tasks (DXC₁₄, DXC₅, and TXC), segmentation tasks (VFS, LXS, and PXS), and PE detection task (ECC), a learning rate of 2e-4, 1e-3, and 4e-4, were utilized, respectively.

FIG. 5 presents Table 3 (element 501) showing the evaluation of iNat2021 mini dataset on segmentation medical tasks, according to described embodiments. Even with less than half number of pre-training samples, iNat2021 mini achieves equal or superior performance over ImageNet counterpart. Best performance is bolded and second best is underlined.

Ablation study on iNat2021 mini dataset—Experiments further investigated the capability of pre-trained models on fine-grained datasets in capturing fine-grained details by examining iNat2021 mini dataset for segmentation tasks. Specifically, iNat2021 mini contains 500K images, which is less than half compared to ImageNet. The results in Table 3 indicate that even with fewer training data, iNat2021 achieves equal or better performance than ImageNet counterpart. This observation suggests that the superior performance of iNat2021 over ImageNet pre-trained model in segmentation tasks should be attributed to the fine-grained nature of data rather than larger pre-training size.

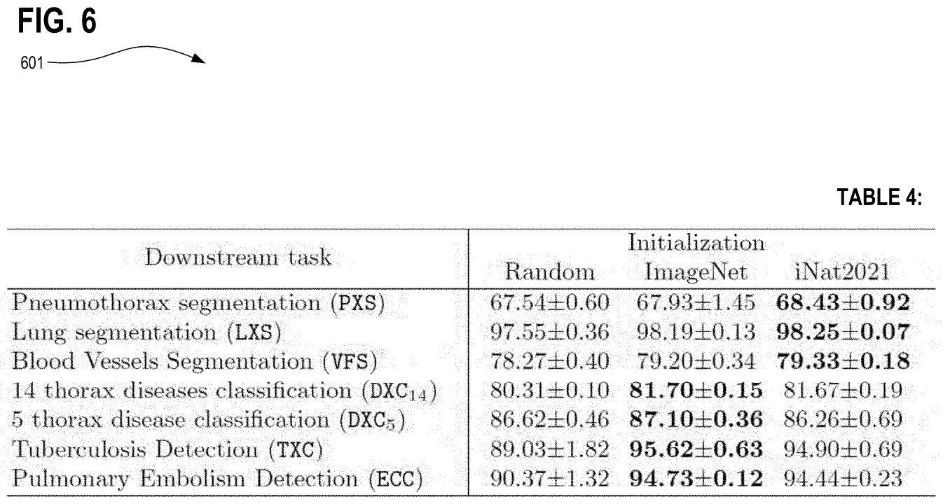

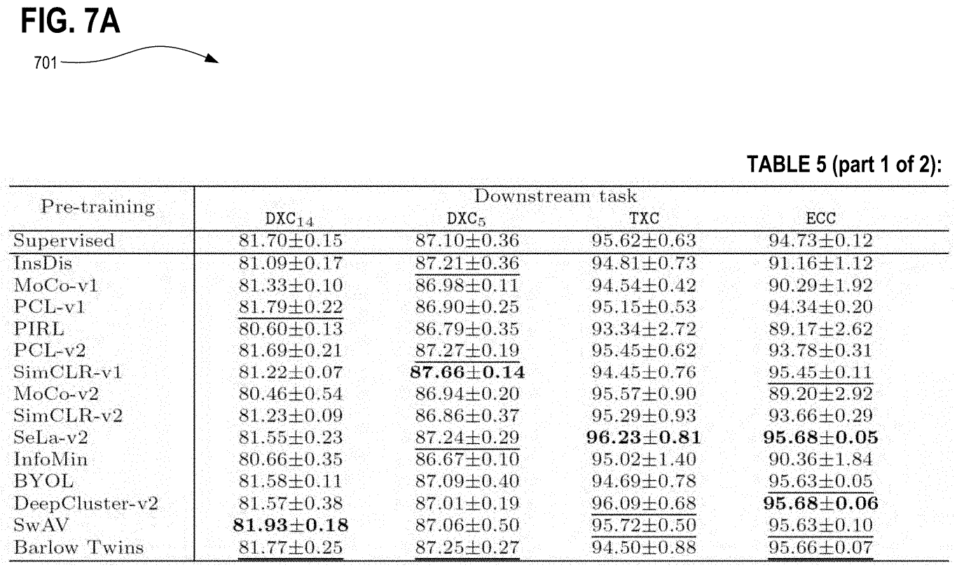

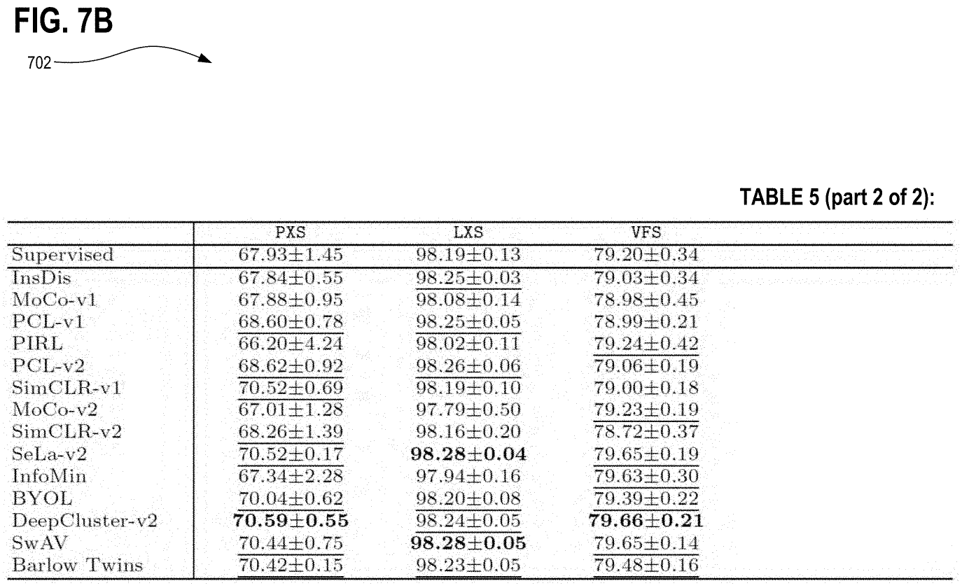

Tabular results—With reference to Table 5, tabulated results of different experiments are reported. The results of the graphs depicted by FIGS. 1A, 1B, and 1C are presented at Table 4 as set forth at FIG. 6, element 601, and the results of the graphs depicted by FIGS. 3A, 3B, 3C, 3D, 3E, 3F, and 3G are presented at Table 5 as set forth at FIG. 7, element 701.

FIG. 6 presents Table 4 (element 601) demonstrating benchmarking transfer learning from supervised iNat2021 and ImageNet models on seven medical tasks, according to described embodiments. Pre-trained models on iNat2021 are better suited for segmentation tasks (e.g., LXS, VFS, and PXS), while pre-trained models on ImageNet prevail on classification tasks (e.g., DXC₁₄, DXC₅, TXC, and ECC). The best model in each application is bolded.

FIG. 7 presents Table 5 (element 701) demonstrating benchmarking transfer learning from fourteen (14) self-supervised ImageNet pre-trained models on seven (7) medical tasks, according to described embodiments. Notably, self-supervised ImageNet models outperform supervised ImageNet models. The best model is bolded, and all the other models that outperform supervised baseline are underlined.

Convergence Time Analysis—Transfer learning attracts great attention since it improves the target performance and accelerates the model convergence when compared to training from scratch. In that respect, a good pre-trained model should yield better target performance with less training time. Therefore, the pre-trained models were further evaluated in terms of accelerating the training process of various medical tasks. Further provided are the training time results for each of the three groups of experiments. The early-stop technique was utilized in all target tasks, and the results report the average number of training epochs over ten (10) runs for each model.

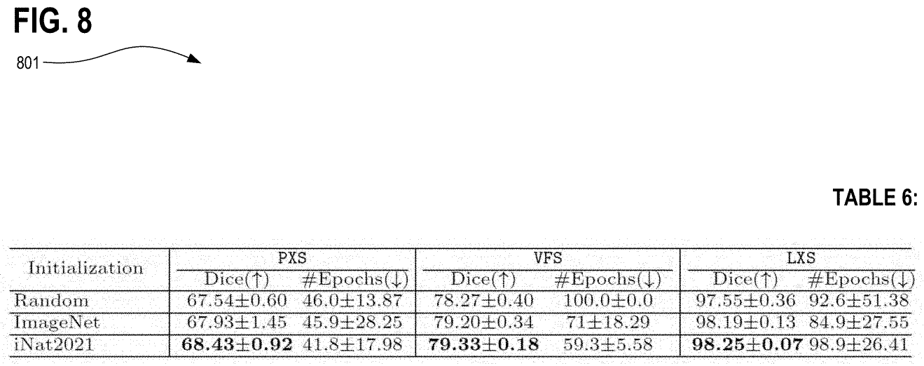

Supervised ImageNet model vs. supervised iNat2021 model—Further provided are the training times of the segmentation tasks in which the iNat2021 model outperforms its ImageNet counterpart. The results in Table 6 as set forth at FIG. 8, element 801, indicate that fine-tuning from the iNat2021 model provides higher performance in all segmentation tasks and considerably accelerates the training process in two out of three tasks in comparison to the ImageNet counterpart.

FIG. 8 presents Table 6 (element 801) demonstrating that fine-tuning from the iNat2021 model provides higher performance in all segmentation tasks and considerably accelerates the training process in two out of three tasks in comparison to the ImageNet counterpart, according to described embodiments. The average performance and number of training epochs over ten (10) runs are both reported for each model in each target task. The best performance in each task is bolded.

Supervised ImageNet model vs. self-supervised ImageNet models—Further demonstrated is a comparison of the training time of the top four self-supervised ImageNet models (based on the overall performances in different target tasks) to the supervised ImageNet model in three target tasks, including classification and segmentation. To provide a comprehensive evaluation, also included are results for training target models from scratch. The results in Table 7 as set forth at FIG. 9, element 901, demonstrate that fine-tuning from the best self-supervised models in each target task provide significantly better or equivalent performance and remarkably accelerate the training process in comparison to the supervised counterpart. Specifically, in DXC₁₄ task, SwAV and Barlow Twins achieve superior performance with significantly less number of training epochs compared to supervised ImageNet model. Similarly, in PXS task, SeLa-v2, DeepCluster-v2, and SwAV outperform supervised ImageNet model in terms of both performance and training time. Furthermore, in VFS task, all the self-supervised models yield higher performance with less training time compared to supervised ImageNet model.

FIG. 9 presents Table 7 (element 901) demonstrating that fine-tuning from the best self-supervised models provide significantly better or equivalent performance and accelerate the training process in comparison to the supervised counterpart, according to described embodiments. The average performance and number of training epochs over ten (10) runs is reported for each model in each target task. The best performance in each task is bolded.

Additionally, considering the principle that a good representation should generalize to multiple target tasks with limited fine-tuning, the experiments further fine-tuned all the models for the same number of training epochs in DXC₅ and ECC (ten and one, respectively). According to the results in Table 5 (refer again to FIG. 7), with the same number of training epochs, the best self-supervised ImageNet models, such as SimCLR-v1, SeLa-v2, and Barlow Twins, achieve superior performance over supervised ImageNet models in both target tasks.

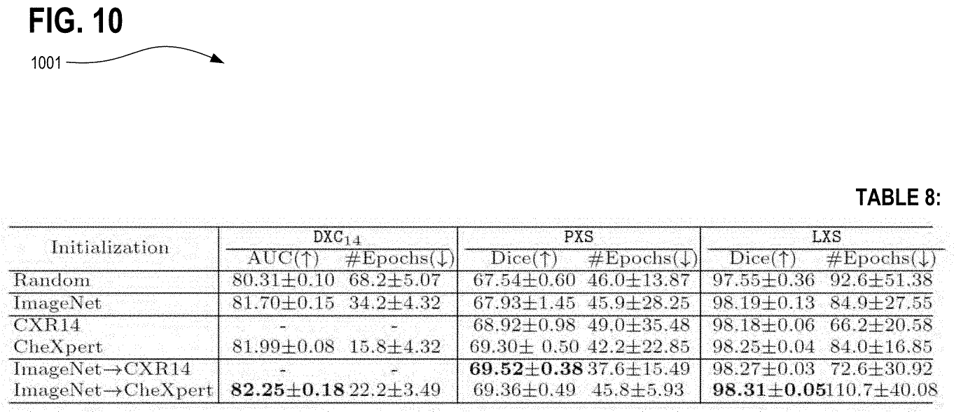

Supervised ImageNet model vs. domain-adapted models—Further demonstrated is a comparison of the training time of the in-domain pre-trained models to ImageNet counterparts. According to the results in Table 8 as set forth at FIG. 10, element 1001, ChestX-ray14 and CheXpert models consistently outperform ImageNet models in terms of convergence time in most cases, and the overall trend showcases the faster convergence of domain-adapted pre-trained models (e.g., ImageNet 4 CheXpert and ImageNet 4 ChestX-ray14) compared to the corresponding ImageNet models.

FIG. 10 presents Table 8 (element 1001) demonstrating that fine-tuning from the domain-adapted pre-trained models provides higher performance in all tasks and speeds up the training process compared to the corresponding ImageNet models in most cases. The average performance and number of training epochs over ten runs is reported for each model in each target task. The best performance in each task is bolded. “CXR14” denotes the ChestX-ray14 dataset. When pre-training and target tasks are the same, transfer learning is not applicable, denoted by the dash (-) symbol.

### Self-Supervised Learning Methods

InsDis: InsDis treats each image as a distinct class and trains a non-parametric classifier to distinguish between individual classes based on noise-contrastive estimation (NCE). InsDis introduces a feature memory bank maintaining a large number of noise samples (referred to as negative samples), to avoid exhaustive feature computing.

MoCo-v1 and MoCo-v2: MoCo-v1 creates two views by applying two independent data augmentations to the same image X, referred to as positive samples Like InsDis, the images other than X are defined as negative samples stored in a memory bank. Additionally, a momentum encoder is proposed to ensure the consistency of negative samples as they evolve during training. Intuitively, MoCo-v1 aims to increase the similarity between positive samples while decreasing the similarity between negative samples. Through simple modifications inspired by SimCLR-v1, such as a non-linear projection head, extra augmentations, cosine decay schedule, and a longer training time to MoCo-v1, MoCo-v2 establishes a stronger baseline while eliminating large training batches.

SimCLR-v1 and SimCLR-v2: SimCLR-v1 is proposed independently following the same intuition as MoCo. However, instead of using special network architectures (e.g., a momentum encoder) or a memory bank, SimCLR-v1 is trained in an end-to-end fashion with large batch sizes. Negative samples are generated within each batch during the training process. In SimCLR-v2, the framework is further optimized by increasing the capacity of the projection head and incorporating the memory mechanism from MoCo to provide more negative samples than SimCLR-v1.

BYOL: Conventional contrastive learning methods such as MoCo and SimCLR relies on a large number of negative samples. As a result, they require either a large memory bank (memory consuming) or a large batch size (computational consuming). On the contrary, BYOL avoids the use of negative pairs by leveraging two encoders, named online and target, and adding a predictor after the projector in the online encoder. BYOL thus maximizes the agreement between the prediction from the online encoder and the features computed from the target encoder. The target encoder is updated with the momentum mechanism to prevent the collapsing problem.

PIRL: Instead of using instance discrimination objectives like InsDis and MoCo, PIRL adapts Jigsaw and Rotation as proxy tasks. Specifically, the positive samples are generated by applying Jigsaw shuffling or rotating images by {0-degrees, 90-degrees, 180-degrees, and 270-degrees }. PIRL defines a loss function based on noise-contrastive estimation (NCE) and uses a memory bank following InsDis. In this paper, experiments only benchmark PIRL with Jigsaw shuffling, which yields better performance than its rotation counterpart.

DeepCluster-v2: DeepCluster learns features in two phases: First, self-labeling, where pseudo labels are generated by clustering data points using the prior representation, thus yielding cluster indexes for each sample. And second, feature-learning, where the cluster index of each sample is used as a classification target to train a model. The two phases are performed repeatedly until the model converges. Rather than classifying the cluster index, DeepCluster-v2 explicitly minimizes the distance between each sample and the corresponding cluster centroid. DeepCluster-v2 finally applies stronger data augmentation, a MLP projection head, a cosine decay schedule, and multi-cropping to improve the representation learning.

SeLa-v2: Similar to clustering methods, SeLa requires a two-phase training (e.g., self-labeling and feature-learning). However, instead of clustering the image instances, SeLa formulates self-labeling as an optimal transport problem, which can be effectively solved by adopting the Sinkhorn-Knopp algorithm. Similar to DeepCluster-v2, the updated SeLa-v2 applies stronger data augmentation, a MLP projection head, a cosine decay schedule, and multi-cropping to improve the representation learning.

PCL-v1 and PCL-v2: PCL-v1 combines contrastive learning and clustering approaches to encode the semantic structure of the data into the embedding space. Specifically, PCL-v1 adopts the architecture of MoCo, and incorporates clustering in representation learning. Similar to clustering-based feature learning, PCL-v1 has self-labeling and feature-learning phases. In self-labeling phase, the features obtained from the momentum encoder are clustered, in which each instance is assigned to multiple prototypes (cluster centroids) with different granularity. In the feature-learning phase, PCL-v1 extends the noise-contrastive estimation (NCE) loss to ProtoNCE loss which can push each sample closer to its assigned prototypes. PCL-v2 is developed by applying the aforementioned techniques to promote the representation learning.

SwAV: SwAV takes advantages of both contrastive learning and clustering techniques. Similar to SeLa, SwAV calculates cluster assignments (codes) for each data sample with the Sinkhorn-Knopp algorithm. However, SwAV performs online cluster assignments, e.g., at the batch level instead of epoch level. Compared with contrastive learning approaches such as MoCo and SimCLR, SwAV “swapped” predicts the codes obtained from one view using the other view rather than comparing their features directly. Additionally, SwAV proposes a multi-cropping strategy, which can be adopted by other methods to consistently improve their performance.

InfoMin: InfoMin hypothesizes that good views (or positive samples) should only share label information with respect to the downstream task while throwing away irrelevant factors, which means optimal views for contrastive representation learning are task-dependent. Following this hypothesis, InfoMin optimizes data augmentations by further reducing mutual information between views.

Barlow Twins: The Barlow Twins method consists of two online encoders that are fed by two augmented views of the same image. The model is trained by making the cross-correlation matrix of two encoders' outputs as close to the identity matrix as possible. As a result, two benefits are realized. First, the similarity between representations of two views is maximized, which is similar to the ultimate goal of contrastive learning, and secondly, the redundancy between the components of two representations is minimized.

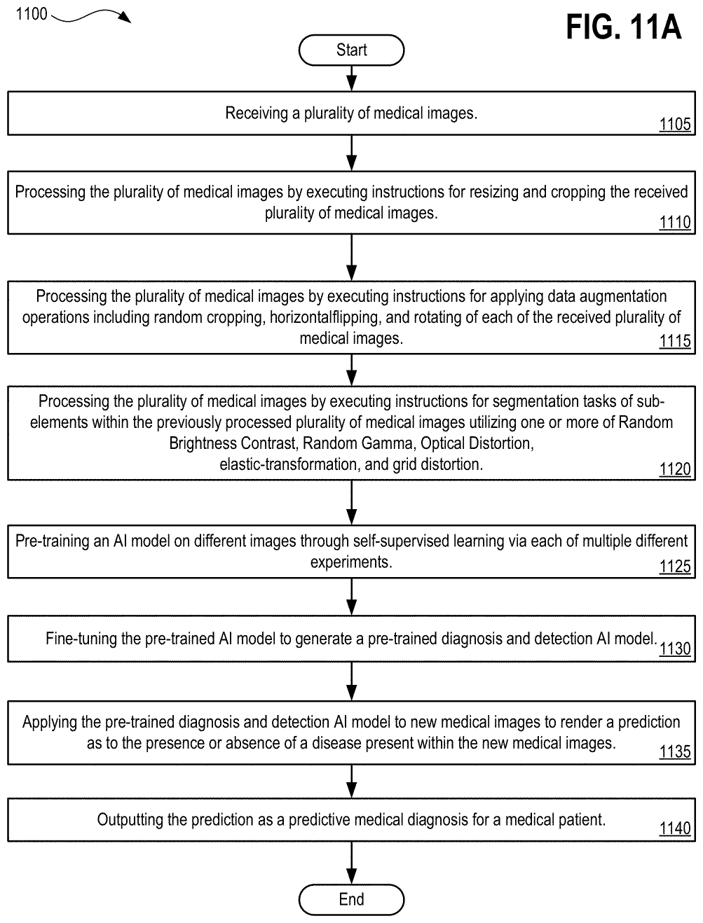

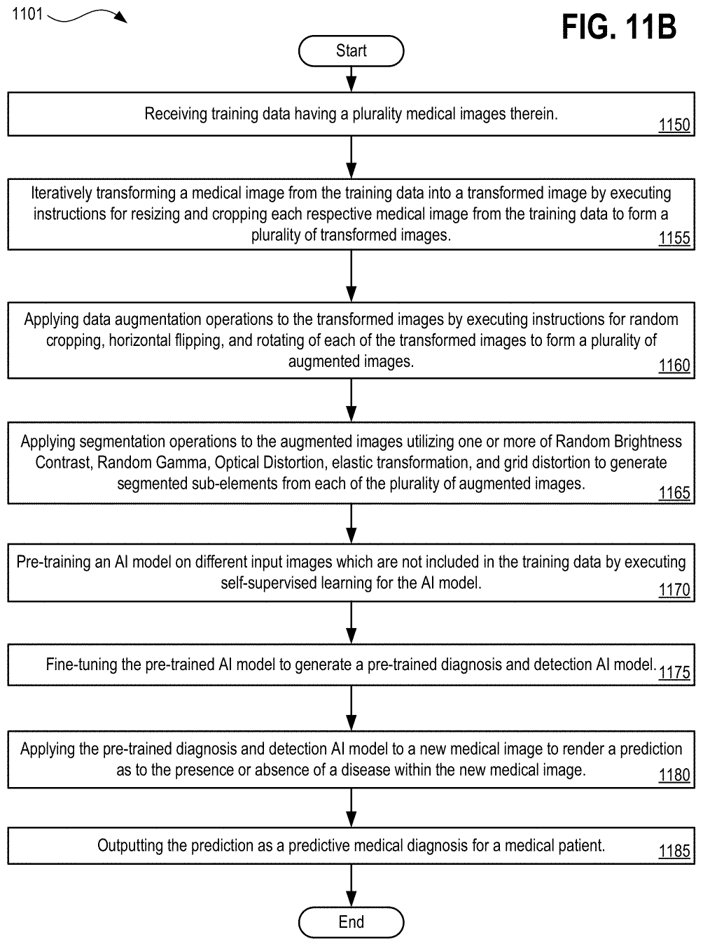

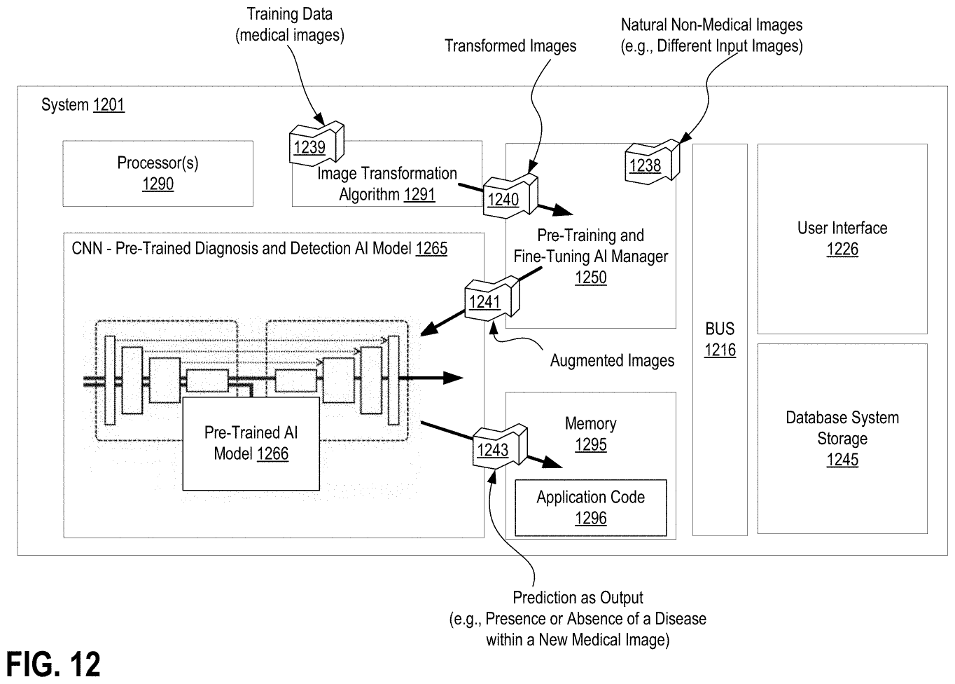

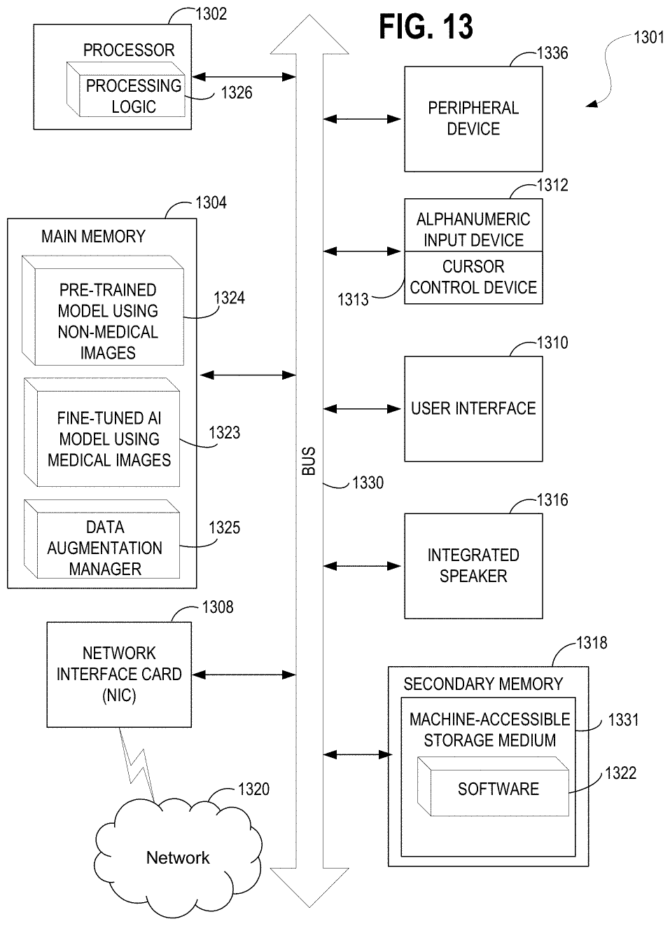

FIGS. 11A and 11B depict flow diagrams illustrating methods 1100 and 1101 for implementing systematic benchmarking analysis to improve transfer learning for medical image analysis, in accordance with disclosed embodiments. Methods 1100 and 1101 may be performed by processing logic that may include hardware (e.g., circuitry, dedicated logic, programmable logic, microcode, etc.), software (e.g., instructions run on a processing device) to perform various operations such as designing, defining, retrieving, parsing, persisting, exposing, loading, executing, operating, receiving, generating, storing, maintaining, creating, returning, presenting, interfacing, communicating, transmitting, querying, processing, providing, determining, triggering, displaying, updating, sending, etc., in pursuance of the systems and methods as described herein. For example, the system 1201 (see FIG. 12) and the machine 1301 (see FIG. 13) and the other supporting systems and components as described herein may implement the described methodologies. Some of the blocks and/or operations listed below are optional in accordance with certain embodiments. The numbering of the blocks presented is for the sake of clarity and is not intended to prescribe an order of operations in which the various blocks must occur.

With reference to the method 1100 depicted at FIG. 11A, there is a method performed by a system specially configured for systematically implementing benchmarking analysis techniques to improve transfer learning for medical image analysis, in the context of processing of medical imaging, in accordance with disclosed embodiments. Such a system may be configured with at least a processor and a memory to execute specialized instructions which cause the system to perform the following operations:

At block 1105, processing logic of such a system receives a plurality of medical images.

At block 1110, processing logic processes the plurality of medical images by executing instructions for resizing and cropping the received plurality of medical images.

At block 1115, processing logic processes the plurality of medical images by executing instructions for applying data augmentation operations including random cropping, horizontal flipping, and rotating of each of the received plurality of medical images.

At block 1120, processing logic processes the plurality of medical images by executing instructions for segmentation tasks of sub-elements within the previously processed plurality of medical images utilizing one or more of Random Brightness Contrast, Random Gamma, Optical Distortion, elastic-transformation, and grid distortion.

At block 1125, processing logic pre-trains an AI model on different images through self-supervised learning via each of multiple different experiments.

At block 1130, processing logic fine-tunes the pre-trained AI model to generate a pre-trained diagnosis and detection AI model.

At block 1135, processing logic applies the pre-trained diagnosis and detection AI model to new medical images to render a prediction as to the presence or absence of a disease present within the new medical images.

At block 1140, processing logic outputs the prediction as a predictive medical diagnosis for a medical patient.

With reference to the method 1101 depicted at FIG. 11B, there is a method performed by a system having at least a processor and a memory therein, wherein the system is specially configured for generating a trained diagnosis and detection AI model via which to analyze a new medical image for a medical patient and then generate as output a medical diagnosis which specifies the presence or absence of a medical disease within the new medical image provided to the trained diagnosis and detection AI model. Such a method operates via the execution of specialized instructions which cause the system to perform the following operations:

At block 1150, processing logic receives training data having a plurality medical images therein.

At block 1155, processing logic iteratively transforms a medical image from the training data into a transformed image by executing instructions for resizing and cropping each respective medical image from the training data to form a plurality of transformed images.

At block 1160, processing logic applies data augmentation operations to the transformed images by executing instructions for random cropping, horizontal flipping, and rotating of each of the transformed images to form a plurality of augmented images.

At block 1165, processing logic applies segmentation operations to the augmented images utilizing one or more of Random Brightness Contrast, Random Gamma, Optical Distortion, elastic transformation, and grid distortion to generate segmented sub-elements from each of the plurality of augmented images.

At block 1170, processing logic pre-trains an AI model on different input images (e.g., such as using natural images or non-medical images) which are not included in the training data by executing self-supervised learning for the AI model.

At block 1175, processing logic fine-tunes the pre-trained AI model to generate a pre-trained diagnosis and detection AI model.

At block 1180, processing logic applies the pre-trained diagnosis and detection AI model to a new medical image to render a prediction as to the presence or absence of a disease within the new medical image.

At block 1185, processing logic outputs the prediction as a predictive medical diagnosis for a medical patient.

According to another embodiment of method 1100-1061, the new medical image constitutes no part of the training data utilized to pre-train or the different input images utilized to fine-tune the pre-trained diagnosis and detection AI model.

According to another embodiment of method 1100-1061, applying the segmentation operations includes applying a segmentation task on a fundoscopic modality (VFS) utilizing random rotation, Gaussian noise, color jittering, and horizontal flips, vertical flips, and diagonal flips.

According to another embodiment of method 1100-1061, fine-tuning the pre-trained AI model to generate a pre-trained diagnosis and detection AI model includes fine-tuning the pre-trained AI model against multiple target tasks to render multi-label classification for the new medical image as part of the prediction outputted as the predictive medical diagnosis.

According to another embodiment of method 1100-1061, fine-tuning the pre-trained AI model to generate a pre-trained diagnosis and detection AI model includes fine-tuning the pre-trained AI model against multiple target tasks to render binary classification for the new medical image as part of the prediction outputted as the predictive medical diagnosis.

According to another embodiment of method 1100-1061, fine-tuning the pre-trained AI model to generate a pre-trained diagnosis and detection AI model includes fine-tuning the pre-trained AI model against multiple target tasks to output pixel-wise segmentation for the new medical image as part of the prediction outputted as the predictive medical diagnosis.

According to another embodiment of method 1100-1061, pre-training the AI model on different input images includes pre-training with fine-grained datasets for transfer learning to medical tasks.

According to another embodiment of method 1100-1061, the fine-grained datasets include deeply embedded visual differences between subordinate classes within local discriminative parts of the medical images received as training data.

According to another embodiment of method 1100-1061, the different input images used for pre-training the AI model constitute natural non-medical images.

According to another embodiment of method 1100-1061, pre-training the AI model on the different input images includes continually pre-training the AI model to minimize a domain gap between the natural non-medical images and the plurality of medical images within the training data.

According to a particular embodiment, there is a non-transitory computer-readable storage medium having instructions stored thereupon that, when executed by a system having at least a processor and a memory therein, the instructions cause the processor to perform operations including: receiving training data having a plurality medical images therein; iteratively transforming a medical image from the training data into a transformed image by executing instructions for resizing and cropping each respective medical image from the training data to form a plurality of transformed images; applying data augmentation operations to the transformed images by executing instructions for random cropping, horizontal flipping, and rotating of each of the transformed images to form a plurality of augmented images; applying segmentation operations to the augmented images utilizing one or more of Random Brightness Contrast, Random Gamma, Optical Distortion, elastic transformation, and grid distortion to generate segmented sub-elements from each of the plurality of augmented images; pre-training an AI model on different input images which are not included in the training data by executing self-supervised learning for the AI model; fine-tuning the pre-trained AI model to generate a pre-trained diagnosis and detection AI model; applying the pre-trained diagnosis and detection AI model to a new medical image to render a prediction as to the presence or absence of a disease within the new medical image; and outputting the prediction as a predictive medical diagnosis for a medical patient.

FIG. 12 shows a diagrammatic representation of a system 1201 within which embodiments may operate, be installed, integrated, or configured. In accordance with one embodiment, there is a system 1201 having at least a processor 1290 and a memory 1295 therein to execute implementing application code 1296. Such a system 1201 may communicatively interface with and cooperatively execute with the benefit of remote systems, such as a user device sending instructions and data, a user device to receive as an output from the system 1201 a specially pre-trained diagnosis and detection AI model 1266 fine-tuned by the pre-training and fine-tuning AI manager 1250 for medical diagnosis tasks on the basis of training data 1239, transformed images 1240 and augmented images 1241. Further depicted is a pre-trained AI model 1266 having been pre-trained using other or different images, such as natural or non-medical images which form no part of the training data which specifically includes only the medical images.

According to the depicted embodiment, the system 1201, includes a processor 1290 and the memory 1295 to execute instructions at the system 1201. The system 1201 as depicted here is specifically customized and configured to systematically generate the pre-trained diagnosis and detection AI model 1266 through the use of improved transfer learning techniques. The training data 1239 is processed through an image transformation algorithm 1291 from which transformed images 1240 are formed or generated. The pre-training and fine-tuning AI manager 1250 may optionally be utilized to refine the AI model to bridge the gap between natural non-medical images and medical images through the application of data augmentation which facilitate improved image segmentation followed by the fine-tuning procedures.

According to a particular embodiment, there is a specially configured system 1201 which is custom configured to generate the pre-trained diagnosis and detection AI model 1266 through the use of improved transfer learning techniques. According to such an embodiment, the system 1201 includes: a memory 1295 to store instructions via executable application code 1296; a processor 1290 to execute the instructions stored in the memory 1295; in which the system 1201 is specially configured to execute the instructions stored in the memory via the processor which causes the system to receive training data 1239 having a plurality medical images therein; iteratively transforming a medical image from the training data 1239 into a transformed image 1240 by executing instructions for resizing and cropping each respective medical image from the training data at the image transformation algorithm 1291 component so as to form a plurality of transformed images 1240; applying data augmentation operations to the transformed images by executing instructions for random cropping, horizontal flipping, and rotating of each of the transformed images to form a plurality of augmented images 1241; applying segmentation operations to the augmented images 1241 utilizing one or more of Random Brightness Contrast, Random Gamma, Optical Distortion, elastic transformation, and grid distortion to generate segmented sub-elements from each of the plurality of augmented images 1241; pre-training an AI model 1266 on different input images 1238 which are not included in the training data 1239 by executing self-supervised learning for the AI model 1266; fine-tuning the pre-trained AI model to generate a pre-trained diagnosis and detection AI model 1265; applying the pre-trained diagnosis and detection AI model 1265 to a new medical image to render a prediction 1243 as output which indicates the presence or absence of a disease within the new medical image; and outputting the prediction 1243 as a predictive medical diagnosis for a medical patient.

According to another embodiment of the system 1201, a user interface 1211 communicably interfaces with a user client device remote from the system and communicatively interfaces with the system via a public Internet.

Bus 1211 interfaces the various components of the system 1201 amongst each other, with any other peripheral(s) of the system 1201, and with external components such as external network elements, other machines, client devices, cloud computing services, etc. Communications may further include communicating with external devices via a network interface over a LAN, WAN, or the public Internet.

FIG. 13 illustrates a diagrammatic representation of a machine 1301 in the exemplary form of a computer system, in accordance with one embodiment, within which a set of instructions, for causing the machine/computer system to perform any one or more of the methodologies discussed herein, may be executed.

In alternative embodiments, the machine may be connected (e.g., networked) to other machines in a Local Area Network (LAN), an intranet, an extranet, or the public Internet. The machine may operate in the capacity of a server or a client machine in a client-server network environment, as a peer machine in a peer-to-peer (or distributed) network environment, as a server or series of servers within an on-demand service environment. Certain embodiments of the machine may be in the form of a personal computer (PC), a tablet PC, a set-top box (STB), a Personal Digital Assistant (PDA), a cellular telephone, a web appliance, a server, a network router, switch or bridge, computing system, or any machine capable of executing a set of instructions (sequential or otherwise) that specify and mandate the specifically configured actions to be taken by that machine pursuant to stored instructions. Further, while only a single machine is illustrated, the term “machine” shall also be taken to include any collection of machines (e.g., computers) that individually or jointly execute a set (or multiple sets) of instructions to perform any one or more of the methodologies discussed herein.

The exemplary computer system 1301 includes a processor 1302, a main memory 1304 (e.g., read-only memory (ROM), flash memory, dynamic random access memory (DRAM) such as synchronous DRAM (SDRAM) or Rambus DRAM (RDRAM), etc., static memory such as flash memory, static random access memory (SRAM), volatile but high-data rate RAM, etc.), and a secondary memory 1318 (e.g., a persistent storage device including hard disk drives and a persistent database and/or a multi-tenant database implementation), which communicate with each other via a bus 1330. Main memory 1304 includes instructions for executing a pre-trained model having been trained using natural or non-medical images 1324 and a fine-tuned AI model 1323 having been trained and fine-tuned to target tasks using medical images as well as a data augmentation manager 1325 which applies data augmentation operations to generate augmented images, in support of the methodologies and techniques described herein. Main memory 1304 and its sub-elements are further operable in conjunction with processing logic 1311 and processor 1302 to perform the methodologies discussed herein.

Processor 1302 represents one or more specialized and specifically configured processing devices such as a microprocessor, central processing unit, or the like. More particularly, the processor 1302 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor 1302 may also be one or more special-purpose processing devices such as an application-specific integrated circuit (ASIC), a field programmable gate array (FPGA), a digital signal processor (DSP), network processor, or the like. Processor 1302 is configured to execute the processing logic 1311 for performing the operations and functionality which is discussed herein.

The computer system 1301 may further include a network interface card 1308. The computer system 1301 also may include a user interface 1310 (such as a video display unit, a liquid crystal display, etc.), an alphanumeric input device 1312 (e.g., a keyboard), a cursor control device 1318 (e.g., a mouse), and a signal generation device 1311 (e.g., an integrated speaker). The computer system 1301 may further include peripheral device 1336 (e.g., wireless or wired communication devices, memory devices, storage devices, audio processing devices, video processing devices, etc.).

The secondary memory 1318 may include a non-transitory machine-readable storage medium or a non-transitory computer readable storage medium or a non-transitory machine-accessible storage medium 1331 on which is stored one or more sets of instructions (e.g., software 1322) embodying any one or more of the methodologies or functions described herein. The software 1322 may also reside, completely or at least partially, within the main memory 1304 and/or within the processor 1302 during execution thereof by the computer system 1301, the main memory 1304 and the processor 1302 also constituting machine-readable storage media. The software 1322 may further be transmitted or received over a network 1320 via the network interface card 1308.

While the subject matter disclosed herein has been described by way of example and in terms of the specific embodiments, it is to be understood that the claimed embodiments are not limited to the explicitly enumerated embodiments disclosed. To the contrary, the disclosure is intended to cover various modifications and similar arrangements as are apparent to those skilled in the art. Therefore, the scope of the appended claims is to be accorded the broadest interpretation so as to encompass all such modifications and similar arrangements. It is to be understood that the above description is intended to be illustrative, and not restrictive. Many other embodiments will be apparent to those of skill in the art upon reading and understanding the above description. The scope of the disclosed subject matter is therefore to be determined in reference to the appended claims, along with the full scope of equivalents to which such claims are entitled.

What is claimed is:

1. A system comprising:
a memory to store instructions;
a set of one or more processors;
a non-transitory machine-readable storage medium that provides instructions that, when executed by the set of one or more processors, the instructions stored in the memory are configurable to cause the system to perform operations comprising:
receiving training data having a plurality medical images therein;
iteratively transforming a medical image from the training data into a transformed image by executing instructions for resizing and cropping each respective medical image from the training data to form a plurality of transformed images;
applying data augmentation operations to the transformed images by executing instructions for random cropping, horizontal flipping, and rotating of each of the transformed images to form a plurality of augmented images;
applying segmentation operations to the augmented images utilizing one or more of Random Brightness Contrast, Random Gamma, Optical Distortion, elastic transformation, and grid distortion to generate segmented sub-elements from each of the plurality of augmented images;
pre-training an AI model on different input images which are not included in the training data by executing self-supervised learning for the AI model;
fine-tuning the pre-trained AI model to generate a pre-trained diagnosis and detection AI model;
applying the pre-trained diagnosis and detection AI model to a new medical image to render a prediction as to the presence or absence of a disease within the new medical image; and
outputting the prediction as a predictive medical diagnosis for a medical patient.

2. The system of claim 1, wherein the new medical image constitutes no part of the training data utilized to pre-train or the different input images utilized to fine-tune the pre-trained diagnosis and detection AI model.

3. The system of claim 1, wherein applying the segmentation operations comprises applying a segmentation task on a fundoscopic modality (VFS) utilizing random rotation, Gaussian noise, color jittering, and horizontal flips, vertical flips, and diagonal flips.

4. The system of claim 1, wherein fine-tuning the pre-trained AI model to generate a pre-trained diagnosis and detection AI model comprises fine-tuning the pre-trained AI model against multiple target tasks to render multi-label classification for the new medical image as part of the prediction outputted as the predictive medical diagnosis.

5. The system of claim 1, wherein fine-tuning the pre-trained AI model to generate a pre-trained diagnosis and detection AI model comprises fine-tuning the pre-trained AI model against multiple target tasks to render binary classification for the new medical image as part of the prediction outputted as the predictive medical diagnosis.

6. The system of claim 1, wherein fine-tuning the pre-trained AI model to generate a pre-trained diagnosis and detection AI model comprises fine-tuning the pre-trained AI model against multiple target tasks to output pixel-wise segmentation for the new medical image as part of the prediction outputted as the predictive medical diagnosis.

7. The system of claim 1, wherein pre-training the AI model on different input images comprises pre-training with fine-grained datasets for transfer learning to medical tasks.

8. The system of claim 7, wherein the fine-grained datasets include deeply embedded visual differences between subordinate classes within local discriminative parts of the medical images received as training data.

9. The system of claim 1:
wherein the different input images used for pre-training the AI model constitute natural non-medical images; and
wherein pre-training the AI model on the different input images comprises continually pre-training the AI model to minimize a domain gap between the natural non-medical images and the plurality of medical images within the training data.

10. A computer-implemented method executed by a system having at least a processor and a memory therein, wherein the method comprises:
receiving training data having a plurality medical images therein;
iteratively transforming a medical image from the training data into a transformed image by executing instructions for resizing and cropping each respective medical image from the training data to form a plurality of transformed images;
applying data augmentation operations to the transformed images by executing instructions for random cropping, horizontal flipping, and rotating of each of the transformed images to form a plurality of augmented images;
applying segmentation operations to the augmented images utilizing one or more of Random Brightness Contrast, Random Gamma, Optical Distortion, elastic transformation, and grid distortion to generate segmented sub-elements from each of the plurality of augmented images;
pre-training an AI model on different input images which are not included in the training data by executing self-supervised learning for the AI model;
fine-tuning the pre-trained AI model to generate a pre-trained diagnosis and detection AI model;
applying the pre-trained diagnosis and detection AI model to a new medical image to render a prediction as to the presence or absence of a disease within the new medical image; and
outputting the prediction as a predictive medical diagnosis for a medical patient.

11. The computer-implemented method of claim 10, wherein the new medical image constitutes no part of the training data utilized to pre-train or the different input images utilized to fine-tune the pre-trained diagnosis and detection AI model.

12. The computer-implemented method of claim 10, wherein applying the segmentation operations comprises applying a segmentation task on a fundoscopic modality (VFS) utilizing random rotation, Gaussian noise, color jittering, and horizontal flips, vertical flips, and diagonal flips.

13. The computer-implemented method of claim 10, wherein fine-tuning the pre-trained AI model to generate a pre-trained diagnosis and detection AI model comprises one of:
fine-tuning the pre-trained AI model against multiple target tasks to render multi-label classification for the new medical image as part of the prediction outputted as the predictive medical diagnosis; or
fine-tuning the pre-trained AI model against multiple target tasks to render binary classification for the new medical image as part of the prediction outputted as the predictive medical diagnosis.

14. The computer-implemented method of claim 10, wherein fine-tuning the pre-trained AI model to generate a pre-trained diagnosis and detection AI model comprises fine-tuning the pre-trained AI model against multiple target tasks to output pixel-wise segmentation for the new medical image as part of the prediction outputted as the predictive medical diagnosis.

15. The computer-implemented method of claim 10:
wherein pre-training the AI model on different input images comprises pre-training with fine-grained datasets for transfer learning to medical tasks; and
wherein the fine-grained datasets include deeply embedded visual differences between subordinate classes within local discriminative parts of the medical images received as training data.

16. The computer-implemented method of claim 10:
wherein the different input images used for pre-training the AI model constitute natural non-medical images; and
wherein pre-training the AI model on the different input images comprises continually pre-training the AI model to minimize a domain gap between the natural non-medical images and the plurality of medical images within the training data.

17. Non-transitory computer readable storage media having instructions stored thereupon that, when executed by a system having at least a processor and a memory therein, the instructions cause the processor to perform operations including:
receiving training data having a plurality medical images therein;
iteratively transforming a medical image from the training data into a transformed image by executing instructions for resizing and cropping each respective medical image from the training data to form a plurality of transformed images;
applying data augmentation operations to the transformed images by executing instructions for random cropping, horizontal flipping, and rotating of each of the transformed images to form a plurality of augmented images;
applying segmentation operations to the augmented images utilizing one or more of Random Brightness Contrast, Random Gamma, Optical Distortion, elastic transformation, and grid distortion to generate segmented sub-elements from each of the plurality of augmented images;
pre-training an AI model on different input images which are not included in the training data by executing self-supervised learning for the AI model;
fine-tuning the pre-trained AI model to generate a pre-trained diagnosis and detection AI model;
applying the pre-trained diagnosis and detection AI model to a new medical image to render a prediction as to the presence or absence of a disease within the new medical image; and
outputting the prediction as a predictive medical diagnosis for a medical patient.

18. The computer-implemented method of claim 10, wherein fine-tuning the pre-trained AI model to generate a pre-trained diagnosis and detection AI model comprises one of:
fine-tuning the pre-trained AI model against multiple target tasks to render multi-label classification for the new medical image as part of the prediction outputted as the predictive medical diagnosis; or
fine-tuning the pre-trained AI model against multiple target tasks to render binary classification for the new medical image as part of the prediction outputted as the predictive medical diagnosis.

19. The computer-implemented method of claim 10:
wherein pre-training the AI model on different input images comprises pre-training with fine-grained datasets for transfer learning to medical tasks; and
wherein the fine-grained datasets include deeply embedded visual differences between subordinate classes within local discriminative parts of the medical images received as training data.

20. The computer-implemented method of claim 10:
wherein the different input images used for pre-training the AI model constitute natural non-medical images; and
wherein pre-training the AI model on the different input images comprises continually pre-training the AI model to minimize a domain gap between the natural non-medical images and the plurality of medical images within the training data.
